(19) United States
(12) Patent Application Publication
(10) Pub. No.: US 2015/0359930 A1
(43) Pub. Date: Dec. 17, 2015

(54) COATED INTRAOCULAR LENS AND ITS MANUFACTURE

(71) Applicant: LENSWISTA AG, Berlin (DE)

(72) Inventors: Thomas KORDICK, Goldbach (DE); Martin GORNE, Hamburg (DE); Guido SCHROTER, Berlin (DE)

(21) Appl. No.: 14/765,052

(22) PCT Filed: Feb. 3, 2014
(86) PCT No.: PCT/EP2014/000278

§ 371 Date: Jul. 31, 2015

(30) Foreign Application Priority Data
Feb. 1, 2013 (EP) 13000513.5
Dec. 27, 2013 (PK) 925/2013

(51) Int. Cl. A61L 27/34; B05D 3/04; B05D 1/00; A61F 2/16; A61L 27/18

(52) U.S. Cl. CPC A61L 27/34; A61F 2/16; A61L 27/18; B05D 1/62; B05D 3/0446

(57) ABSTRACT

An intraocular lens has a hydrophobic lens body (1) made of silicone, at the surface of which a hydrophilic layer (2) made of polyacrylate is provided, wherein the layer (2) is a PECVD/CVD-layer having a water contact angle of less than 10°. A process for hydrophilizing the surfaces of the intraocular lens includes steps for PECVD-pre-coating the pre-treated lens surfaces, and for c) CVD-follow-up-coating the so pre-coated lens surfaces.

The present invention relates to a coated intraocular lens, IOL, and a method for its manufacture.

Such lenses are employed in particular after degradation of the natural eye lens to replace same, by way of implantation in the course of a cataract operation. Known lens bodies consist of a hydrophobic material, in particular copolymers, which contain acrylate or methacrylate. To reduce tackiness, it is also known to add fluorinated acrylate or methacrylate to the lens material (WO 2007/062864). It is further known to use, for the intraocular lens, a lens material having a high index of refraction, in order to enable a small lens thickness. Such lenses, when foldable or rollable, may be implanted into the eye through relatively small incisions by means of injectors as known from U.S. Pat. No. 6,355,046 B2.

Because of the difference of the index of refraction of the lens material as compared to the medium surrounding the eye, namely the chamber water in the anterior chamber of the eye, and the vitreous body at the lens back face, at the interfaces light reflections result. This is the more pronounced, the higher is the difference of the index of refraction of the lens material to that of the surrounding medium.

The present invention also relates to the surface treatment of workpieces on the basis of biocompatible materials, and in particular relates to a permanent hydrophilation of surfaces of such workpieces, in particular IOLs, by means of plasma enhanced chemical vapour deposition (PECVD) and subsequent chemical vapour deposition (CVD).

There are high requirements to the biological compatibility of workpieces intended for temporary or permanent use in human or animal organs, such as e.g. intraocular lenses, in order to avoid inflammatory processes. In order to ensure a high biocompatibility, the materials used for manufacturing such workpieces have properties predestining them both for the intended use as also to the tissue contact associated therewith. The biocompatibility of materials is determined to a large extent by their surface properties. For IOLs, a hydrophilic surface is decisive for a good biocompatibility.

A biocompatible hydrophilation of surfaces of polymeric biomaterials may be achieved by a modification of the polymeric surface by means of plasma oxidation, as described e.g. in the international application WO 99/57177. It turned out, however, that such hydrophilized surfaces are not sufficiently long-time stable.

A more permanent hydrophilation of polymer biomaterial surfaces is achieved by coating same with a hydrophilic biocompatible material. In order to manufacture hydrophilic surfaces on contact lens made of polymethylmethacrylate (PMMA), in the patent document U.S. Pat. No. 5,080,924 e. g. a plasma coating process for graft polymerising the surfaces with polyacrylic acid has been suggested. The graft polymerised PAA-surfaces showed a contact angle of water in the range 35 to 50 degrees and are too large for a sufficient wetting of the material's surface. For a further reduction of the contact angle, the coating needs to be post-treated, e.g. by applying a further biocompatible material different from acrylic acid, which cross-links to the polyacrylic acid. Such a process involving coating plural layers requires a higher apparative effort and also results in longer coating times, reducing efficiency.

Starting out from what has been described above, it is therefore desirable to provide a less complex coating of polymeric biomaterials, in particular of IOLs, which enables a long-term stable surface hydrophilation with water contact angles of 15 Grad or less.

Such a lens has a hydrophobic lens body, at the surface of which a hydrophilic layer is provided. In embodiments, the lens body consists of a hydrophobic, foldable or rollable polymer material such as silicone rubber. The hydrophilic layer consists of a hydrophilic (meth)acrylate (in its broadest sense, i.e. including the acid, its salts, and its esters) with good tissue and blood compatibility. This coating prevents the adhering of fibrin and cells and thereby counteract a post-operative membrane formation (secondary cataract).

The hydrophobic lens material suitably is one which takes up less than 5 Vol % water.

The lens bodies made of silicone rubber can be made in a molding process. Herein, the roughness of the mold surfaces translates into a roughness of the lens surfaces. By applying the hydrophilic coating onto this surface, the roughnesses are evened out and light diffraction is substantially avoided.

The index of refraction of the hydrophilic coating is suitably selected so as to be nearly that of the lens material and nearly that of the surrounding medium in the eye, i.e. the chamber water and the vitreous body. This means that the index of refraction may, in embodiments, be selected from between n=1.336 (chamber water) or 1.338 (vitreous body) and n=1.56 (that of the polymer material known from WO 2007/062864 A2). Where the index of refraction of the hydrophilic acrylate layer is nearly that of the hydrophobic lens material, from which the lens body is made, and the surrounding medium in the eye, namely the chamber water and the vitreous body, a sufficiently smooth light optical transition between the chamber water, the lens body and the vitreous body is achieved, thereby reducing or avoiding light reflection and light diffraction through micro roughnesses.

The hydrophilic coating also improves on the gliding property of the lens for implanting it by means of an injector. Such injectors are known e.g. from U.S. Pat. No. 6,355,046 B2 and serve for holding or rolling the lens to be implanted. On implanting, the folded or rolled lens is implanted into the eye through a tube which is inserted through a minimal incision in the eye.

If desired, the intraocular lens, IOL, can be stored in a disposable injector. Because of the hydrophilic coating on the intraocular lens and, optionally, on the inner wall of the injector tube, an improved gliding property and thus an easier implanting of the intraocular lens is achieved.

The coating comprises a process for hydrophilizing the surfaces of intraocular lenses, in which the process includes a step (a) of cleaning and activating the lens surface in the course of a pre-treatment with a high frequency plasma• formed on the basis of an inert gas; a step (b) of pre-coating the pre-treated lens surfaces with polyacrylic acid using a high frequency plasma generated on the basis of a gas mixture, wherein the gas mixture is composed of an inert gas and a first gas formed of biocompatible, polymerisable, carboxy group-containing monomers, and a step (c) of follow-up coating the pre-coated lens surfaces using a second gas containing mainly acrylic acid or acrylic anhydride monomers. The follow-up coating involves no plasma.

The coating further comprises providing an IOL with a hydrophilizing surface coating of polyacrylic acid, obtainable according to a process including the step specified above, wherein the contact angle of water on the lens surface coated with polyacrylic acid has a value in the range of 2 to less than 10 degrees, or in the range below 2 degrees (and larger than 0).

The IOLs coated with the specified process have a long-time stable hydrophilic surface with excellent wettability, which in contact with body tissue results in a good bio compatibility, whereby irritations of the eye are met with less frequent with accordingly coated IOLs.

In embodiments of the process, the biocompatible, polymerisable carboxy-group-containing monomers forming the first gas are selected from (meth)acrylic acid and (meth)acrylic anhydride, whereby in the high frequency plasma a large proportion of acrylic acid monomers is generated, which attach to the lens surface activated in step (a) of the process forming covalent bonds.

In other embodiments, the gas used in step (a) of the process for generating the high frequency plasma contains the first gas in an amount corresponding to a partial pressure of less than one tenth of the partial pressure of the inert gas, so that an efficient cleaning and activating of the lens surface is ensured. Alternatively, this step is essentially dispensed with, and there is no initial plasma treatment step in the absence of the first gas, or with less than 10% of the total gas pressure being due to the polymerisable monomers. In some embodiments, in the initial plasma treatment, not less than one part in eight, or one part in six of the total gas pressure is due to the partial pressure of the monomers.

In order to achieve a stable attachment of acrylic acid monomers to the lens surface, in embodiments, in step (b) a gas mixture is used, in which the partial pressure of the first gas is at least one fourth of, and maximally twice the partial pressure of the inert gas.

With a view to obtaining a dense and stable polyacrylic acid coating, the partial pressure of the inert gas in the second gas used in step (c) is, in embodiments, less than one tenth of the partial pressure of the acrylic acid monomer forming gas.

In embodiments, argon is used as the inert gas.

For an effective control of the pre-coating process, in embodiments the coating applied in step (b) is monitored by means of a layer thickness control device, and the process terminated upon reaching a layer thickness value selected from the range 50 to 400 Å.

In other embodiments, in which a contact angle in the range 2 to less than 10 degrees is achieved, the pressure of the inert gas for the high frequency plasma in step (a) is set to a value in the range about 2 Pa to about 8 Pa (about 15 mTorr to about 60 mTorr) and the pressure of the first gas for the high frequency plasma in step (b) is set to a value in the range about 4 Pa to about 12 Pa (about 30 mTorr to about 90 mTorr).

For fixing the acrylic acid polymer coating on the lens surfaces, embodiments further include a step (cb), comprising throttling or choking the inert gas supply directly after step (b), and supplying a second gas instead. In embodiments, the pressure of the second gas in step (cb) is less than about 40 mPa (about 0.3 mTorr).

In order to promote the attachment and cross-linking of acrylic acid monomers to resp. with the pre-coated lens surface, in embodiments a further step (bc) is carried out, immediately after step (b) or, if executed, after step (cb), which further step is a switching-off of the high frequency plasma, an interrupting, reducing or throttling the inert gas supply, and a supplying of the second gas, wherein the pressure of the second gas in step (c) is between about 0.13 kPa and about 0.8 kPa (about 1.5 to about 6 Torr).

In order to improve on the bio-compatibility, in embodiments step (c) is followed by a further step (d) of removing water soluble components from the hydrophilizing layer by means of rinsing the coated IOL in a hydrophilic solvent such as e.g. an isotonic saline solution or in demineralized water, optionally followed by vacuum drying.

In further preferred embodiments, the IOL comprises, at least at its surface, a material which is formed mainly or substantially of a silicone, in particular poly(dimethylsiloxane), or a silicone hydrogel.

In embodiments, the lenses are silicone IOLs. The hydrophilizing surface coating of these lenses, provided by the process, is comprised of a PAA-layer with an average thickness of about 5 μm to about 40 μm.

Further features of the invention are apparent from the following description of embodiments in conjunction with the claims and the drawings. The invention is not limited to the described embodiments, but is defined by the enclosed claims. In particular, individual features of embodiments of the invention may be realized in a different number or combination than in the examples below. In the following explanation of embodiments, it is made reference to the appended drawings, which show:

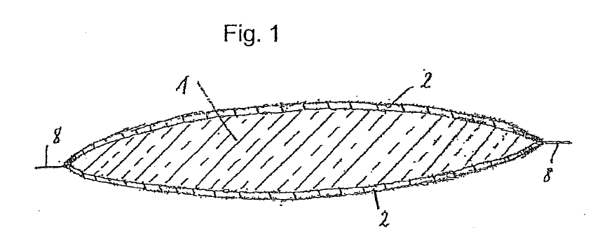

FIG. 1 schematically a cross-sectional view of an intraocular lens according to an embodiment of the invention;

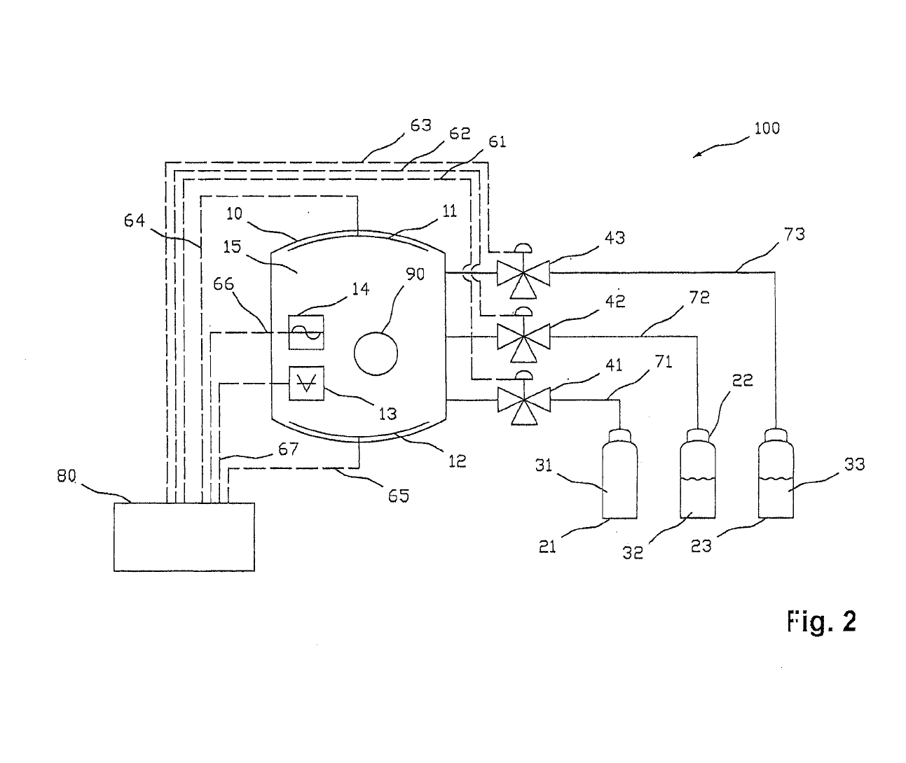

FIG. 2 a schematic view to illustrate a system for the biocompatible coating of polymeric biomaterials,

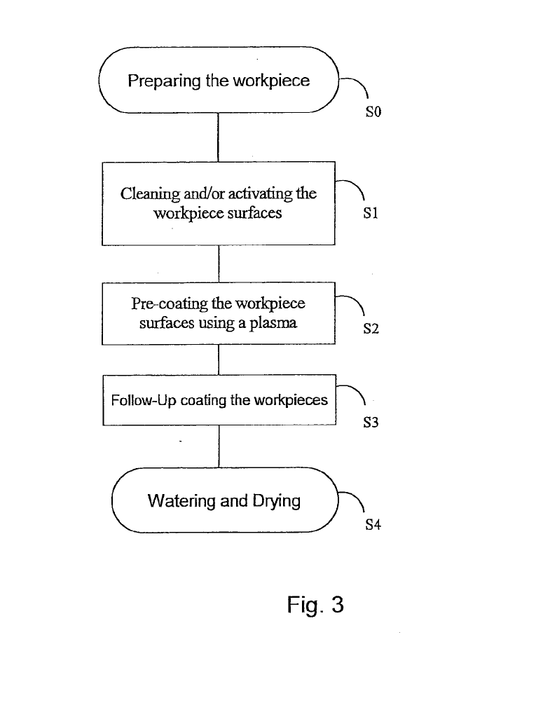

FIG. 3 a flow diagram to illustrate the important steps for the coating of polymeric biomaterials with polyacrylic acid, and

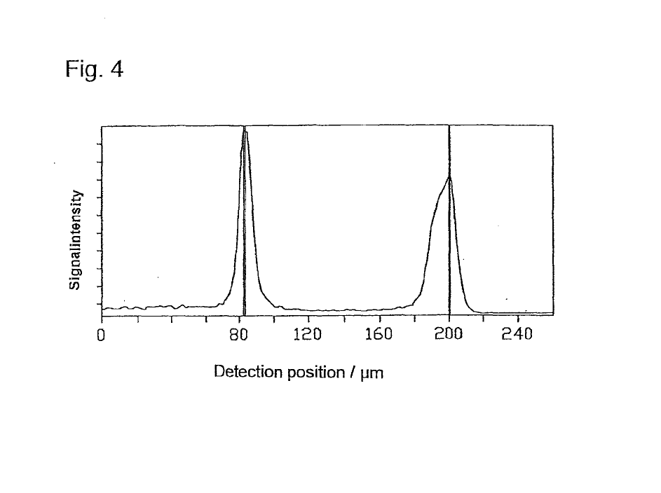

FIG. 4 a fluorescence diagram to illustrate the layer thickness achieved with the process according to FIG. 3.

The embodiment of an intraocular lens in FIG. 1 shows a lens body 1 of hydrophobic silicone. In some embodiments, the lens material is configured for the lens body 1 to be foldable or rollable. The surface of the lens body 1 has a micro roughness stemming form the molding process. Onto the micro-rough surface of the lens body 1, a hydrophilic layer 2 of a hydrophilic acrylate (in the wider sense) is applied. The coating is applied with the coating procedure described below. At the rim of the optical lens body, haptics 8 in the shape of Form of filaments or struts or in the shape of a supporting frame surrounding the lens body 1 wholly or in part may be provided.

Onto the lens body 1 of a hydrophobic material, e.g. silicone, after activating the surface of the lens body 1 the hydrophilic layer 2 is applied. The activating of the surface is made by plasma activation, e. g. using a nitrogen or argon plasma. Onto the activated surface, the monomer of the hydrophilic acrylate is applied. Subsequently, the surface if washed and dried in vacuum at about 35° C. The haptics 8 need not be made from silicone, but may be made of e. g. PMMA, PP or polyimide.

Without wishing to be bound by theory, it is believed that in the process, molecules of the hydrophilic acrylate (including acrylic acid or anhydride) diffuse into the subsurface region of the hydrophobic lens material and may partly cross-link. This process is seemingly enhanced by the activation described above.

The scheme shown in FIG. 2 illustrates important components of a device 100 for coating polymeric workpieces 90 with a material rendering their surfaces hydrophilic. The work pieces may be intraocular lenses, IOLs, and in particular such ones made of a silicone or a silicone hydrogel.

The apparatus 100 comprises an evacuatable recipient 10 with a device for generating a high frequency plasma in the interior 15 of the recipient 10. The device for generating a high frequency plasma is symbolized in the scheme of FIG. 2 by means of two electrodes 11 and 12, but is not limited to the use of electrodes. In FIG. 2, for the sake of clarity and conciseness, only those components are depicted which are deemed to be required for understanding the invention. Such component as e.g. pumps for evacuating the recipient 10, which are required for operating the apparatus but are irrelevant for understanding the invention, are deemed present despite not being shown in the drawing. With the interior 15 of the recipient 10, at least a vacuum or low pressure gauge 13 and a coating application measuring device 14, e.g. an oscillating quartz, are associated.

The coating apparatus 100 further includes an inert gas reservoir 21 and one or more coating material reservoirs 22 and 23. Each of the reservoirs or reservoir containers 21, 22 and 23 is connected by an associated one of ducts 71, 72 and 73 with the recipient 10 in such a manner that gaseous or vaporized substances kept in the reservoirs or reservoir containers can be guided into the interior 15 of the recipient 10. Control valves 41, 42 and 43 arranged in the ducts 71, 72 and 73 enable control or regulation of the flow of the respective gas or vapour into the recipient 10. In the embodiment shown, the control valves may alternatively be used for venting the reservoirs 21, 22 and 23. In other embodiments, separate valves are employed for this purpose.

The apparatus 100 further includes a control 80, which is adapted for controlling or regulating the coating processes e.g. by means of control leads 61, 62, 63, 64, 65 and signal leads 66 and 67. Depending on the requirements, the control can be adapted for a fully automatic or a semi-automatic coating process, or for selectably fully or semi-automatic coating control. A regulating (partial) control of the apparatus 100 can be realized e. g. using the output signals of sensor devices associated with the interior 15. For example, the valves 41, 42 and 43 may be controlled, using the vacuum or low pressure gauge 13, in such a manner that in the interior 15 of the recipient 10 a desired constant gas or vapour pressure with likewise desired partial pressures is maintained. Furthermore, the control device 80 may be adapted to monitor the building-up of the coating by means of the coating application measuring device 14 and to terminate same when a desired coating thickness is reached. In addition, the control 80 is typically arranged for controlling the high frequency apparatus 11 and 12 in dependence of the process requirements.

The flow diagram 200 of FIG. 3 illustrates the important steps of a process for hydrophilizing lens surfaces by coating with polyacrylic acid. Preferably, polymeric biomaterials are used for manufacturing the lenses 90 or their surface regions, wherein “biomaterial” designates all materials intended and suitable for contact with biological tissue or body fluids.

Subsequent to the preparation of the workpieces 90 in step S0, optionally comprising cleaning the workpieces and arranging same in the recipient 10 as well as subsequently evacuating the recipient, the workpiece surfaces are initially prepared in step S1 for a subsequent coating.

To this end, the recipient 10 loaded with the one or more workpieces is initially evacuated by means of pumps (not shown), preferably to a pressure of maximally 10⁻⁴ mbar (10 mPa). After reaching the desired vacuum pressure, the interior 15 of the recipient is flooded with an inert gas, preferably argon, while continually pumping, .wherein the flow of the inert gas is adjusted to the pumping speed that in the interior 15 of the recipient 10 a constant gas pressure is maintained. The inert gas 31 is supplied to the recipient from an inert gas source 21. In embodiments, the Argon gas pressure is about 25 mTorr (ca. 3,33 Pa). After reaching a stable inert gas pressure in the interior 15 of the recipient, the plasma generator, e. g. a high voltage generator, is switched on, whereby an inert gas plasma is created which surrounds the workpieces 90. The plasma cleans the lens surfaces by removing substances adsorbed thereto and further results in an activation of the lens surfaces by forming ions and free radicals beneficial for the subsequent polymerisation process. In some situations, however, an initial plasma application step in the absence, or substantial absence, of a reactive gas component is dispensible.

If applied, the cleaning and activating effect of this first step S1 may be influenced via the frequency of the gas plasma, the power introduced into the plasma, the activation time of the plasma, and the type of the inert gas used for the plasma, as is generally known. The settings suitable for each individual application case may be determined by the skilled person. In the presently described process Argon is preferred as the inert gas, because it allows an activation of the workpiece surfaces without generating new, undesired compounds. Naturally, other inert gases may be employed instead, such as nitrogen, if leading to comparable results. In an exemplary embodiment, the exposition time to the Argon plasma is about one minute or less.

After this time, the plasma generator may be switched off and the process continued with the first actual coating step S2. Deviating from the above, the plasma employed for the pretreatment of the workpiece may be generated on the basis of a mixture of the inert gas and a reactive component to be used in a subsequent pre-coating step, instead of pure Argon. The partial pressure of the reactive component in the gas mixture should in this case be less than one tenth of the partial pressure of the inert gas.

While transitioning from step S1 to step S2 of the process, the flow of inert gas into the interior of the recipient is preferably maintained or optionally adjusted so that it assumes a value suitable for carrying out step S2. For generating the gas mixture, a coating material gas made up of biocompatible, polymerizable carboxy group-containing monomers in the vapour phase is admixed to the inert gas in the recipient 10. The carboxy group-containing monomers may preferably be acrylic acid or an acrylic acid precursor, such as e. g. (meth)acrylic acid anhydride. The partial pressure P_{eSG} of the first coating material gas in some embodiments is at least one quarter of, and maximally twice the partial pressure P_{IG} of the inert gas. More preferably, the partial pressure ratio P_{eSG}:P_{IG} is selected from the range 1:1 to 1:0.5. E. g., the partial pressure of Argon in some embodiments is 30 mTorr (ca. 4 Pa) at a total pressure of the gas mixture of 45 mTorr (ca. 6 Pa), resulting in a value of the ratio of the Argon partial pressure P_{Ar} to the partial pressure of the first coating material gas partial pressure (reactive component partial pressure) P_{eSG} of 2:1.

As the reactive component for generating the first coating material gas, preferably (meth)acrylic acid anhydride is used, which is vaporized in one of the reservoirs 22 or 23 shown in FIG. 1 and is guided to the interior 15 of the recipient 10 via ducts 72 or 73. The partial pressure of the coating material gas is adjusted via its inflow, in turn controlled through valves 42 or 43. Naturally, instead of (meth)acrylic acid anhydride, (meth)acrylic acid may be used. (Meth)acrylic acid or (meth)acrylic acid anhydride, respectively, are provided in the reservoirs 22 or 23 in liquid form, e. g. in a amount of 150 mL.

In order to prevent polymerisation of acrylic acid or its precursor material, respectively, same may be doted with Cu(I) chloride. Furthermore, the reactive component reservoirs 22 and 23, respectively, after filling are de-aerated until bubbles no longer appear in the reactive component liquid. The vapour pressure of the reactive component liquid at common ambient temperatures of 22 to 25° C. is usually sufficient for forming the first coating material gas.

After adjusting the desired gas mixture and gas mixture pressure, the actual pre-coating process is initiated through starting the plasma generator, whereby acrylic acid monomers excited in the plasma attach to the potentially activated workpiece surface and, in the further course, form a poly(acrylic acid) layer. This plasma enhanced pre-coating phase is maintained until a desired coating thickness is reached. The growth of the coating may be monitored by means of the coating application measuring device 14. In principle, coatings with thicknesses of up to 30 μm may be applied, wherein a respective coating process is terminated once the coating application measuring device 14 indicates the achievement of the desired coating thickness within a given tolerance of e. g. 0.5 to 4 Å. For hydrophilizing IOLs, pre-coatings with thicknesses in the range of as low as about 0.5 to 4 nm have proven suitable. In dependence of the coating thickness to be achieved, the pre-coating phase may take less than 10 minutes, less than 3 minutes or less than 1 minute. The gas supply is preferably maintained unchanged during the plasma coating. The pre-coating process may be terminated by switching off the plasma generator.

Subsequent to the pre-coating step S2, and after switching off the plasma generator, in the follow-up coating step S3 initially the inert gas supply is throttled or interrupted and the precoated workpiece surface is exposed to preferably the full vapour pressure of a reactive component formed of water-free acrylic acid. The vapour pressure of the reactive component should not be less than 5 Torr (ca. 667 Pa). Slightly cooling (but without solidifying!) or cautiously warming the reactive component in the reservoir 22 or 23 may be suitable for adjusting the pressure. The introduction of the reactive component into the recipient 10 at full vapour pressure provides the reactive gas in large amounts, which reacts with reactive centers present on the pre-coated surface and provides a relatively thick poly(acrylic acid) layer (PAA-layer), which may be crystalline.

In FIG. 4, a measurement diagram is shown, from which it may be derived that a PAA-layer produced as described above has a thickness of about 10 μm. For performing the measurement, the hydrophilic PAA-layer was stained with Rhodamin 6G as a fluorescence dye, and the fluorescence measured in dependence of depth by means of confocal microscopy. As may be gathered from the right portion of the fluorescence signal tracks, the hydrophilic layer extends significantly into the depth of the workpiece. The (meniscus) lens measured in FIG. 4 at the position of the measurement has a thickness of 117.5 μm. The resolution of the measurement is 0.6 μm. From the obtained data, it may be derived that a coating thickness on the surfaces of ca. 10±0.6 μm and a penetration depth per side of ca. 15 to 20±0.6 μm was present. The process described above is therefore particularly suitable for the application to silicone IOLs, for which hydrophilicity of the surface, durability of the coating as well as the optical properties thereof are equally important.

After terminating the process in step S4, the coated workpieces 90 may be removed from the recipient and may optionally be subjected to quality control, cleaning, and drying.

Before the coating of an IOL, the lens body, which is hydrophobic, is molded with two mutually opposite convex lens surfaces, which in contact with the vitreous body or chamber water, respectively, provide the required refractive power. The coating is applied to the finished lens surface after its removal from the molding tool through combined PECVD (pre-coating) and CVD (follow-up coating), wherein PECVD indicates “Plasma-Enhanced CVD” and CVD means “Chemical Vapor Deposition” (i.e., without or essentially without action of a plasma).

The process described above enables durable hydrophilisation of the surfaces of intraocular lenses made of silicone (or other workpieces), which in turn allows for an excellent wetting with water and, thereby, a high biocompatibility.

The skilled person will realize that numerous modifications and alterations of the examples described above are possible, without leaving the scope of the appended claims.

1-9. (canceled)

10. A process of hydrophilizing the surfaces of an intraocular lens, the process comprising
(a) pre-treating the lens surfaces for cleaning and activating the lens surfaces in a high frequency plasma formed on the basis of an inert gas,
(b) pre-coating the so-pretreated lens surfaces with polyacrylic acid using a high frequency plasma generated from a gas mixture, wherein the gas mixture is composed of an inert gas and a first gas formed of biocompatible, polymerizable, carboxy group-containing monomers, and
(c) follow-up coating the so-precoated lens surfaces,
wherein the pre-coating according to (b) takes less than 10 minutes, and wherein the follow-up coating according to (c) occurs using a second gas substantially containing acrylic acid or acrylic anhydride monomers.

11. The process of claim 10, wherein the gas used in (a) for generating the high frequency plasma contains the first gas in an amount corresponding to a partial pressure of less than one tenth of the partial pressure of the inert gas.

12. A process of hydrophilizing the surfaces of an intraocular lens, the process comprising
(a) pre-coating lens surfaces with polyacrylic acid using a high frequency plasma generated from a gas mixture, wherein the gas mixture is composed of an inert gas and a first gas formed of biocompatible, polymerizable, carboxy group-containing monomers, and
(b) follow-up coating the so-precoated lens surfaces,
wherein the pre-coating according to (a) takes less than 10 minutes, and wherein the follow-up coating according to (b) occurs using a second gas substantially containing acrylic acid or acrylic anhydride monomers.

13. The process of claim 12, wherein there is no preceding plasma-activation in the absence of the first gas or with less than 10% of the gas formed of the monomers.

14. The process of claim 12, wherein the monomers constituting the first gas are selected from acrylic acid and acrylic acid anhydride.

15. The process of claim 12, wherein in the gas mixture used in (a), the partial pressure of the first gas is at least one fourth of, and maximally twice the partial pressure of the inert gas.

16. The process of claim 12, wherein the partial pressure of the inert gas in the second gas used in (b) is less than one tenth of the partial pressure of the gas formed of acrylic acid or acrylic acid anhydride monomers.

17. The process of claim 12, wherein the intraocular lens is formed from silicone or a silicone hydrogel at least in its surface region.

18. The process of claim 17, wherein the intraocular lens is formed from poly(dimethylsiloxane) at least in its surface region.

19. The process of claim 17, comprising forming the intraocular lens, before coating it, with two mutually opposing convex lens surfaces for providing the required refractive power of the eye.

20. The process of claim 10, wherein the monomers constituting the first gas are selected from acrylic acid and acrylic acid anhydride.

21. The process of claim 10, wherein in the gas mixture used in (b), the partial pressure of the first gas is at least one fourth of, and maximally twice the partial pressure of the inert gas.

22. The process of claim 10, wherein the partial pressure of the inert gas in the second gas used in (c) is less than one tenth of the partial pressure of the gas formed of acrylic acid or acrylic acid anhydride monomers.

23. The process of claim 10, wherein the intraocular lens is formed from silicone or a silicone hydrogel at least in its surface region.

24. The process of claim 23, wherein the intraocular lens is formed from poly(dimethylsiloxane) at least in its surface region.

25. The process of claim 23, comprising forming the intraocular lens, before coating it, with two mutually opposing convex lens surfaces for providing the required refractive power of the eye.
